(19) United States
(12) Patent Application Publication
(10) Pub. No.: US 2024/0074989 A1
(43) Pub. Date: Mar. 7, 2024

(54) ACTINIC KERATOSIS TREATMENT

(71) Applicant: COEGIN PHARMA AB, Lund (SE)

(72) Inventors: Berit JOHANSEN, Lund (SE); Tore DUVOLD, Lund (SE); Astrid Jullumstrø FEUERHERM, Lund (SE); Felicity ASHCROFT, Lund (SE)

(21) Appl. No.: 18/260,160

(22) PCT Filed: Dec. 30, 2021
(86) PCT No.: PCT/EP2021/087848

§ 371 Date: Jun. 30, 2023

(30) Foreign Application Priority Data
Dec. 31, 2020 (GB) 2020843.5

(51) Int. Cl. A61K 31/11; A61P 17/00

(52) U.S. Cl. CPC A61K 31/11; A61P 17/00

(57) ABSTRACT

A compound of formula (I) R—S—CH₂—CO—CF₃ (I) wherein R is a C₁₀₋₂₄ unsaturated hydrocarbon group comprising at least 4 non-conjugated double bonds; or a salt thereof; for use in the treatment of squamous cell carcinoma or actinic keratosis.

### BACKGROUND

This invention relates to the use of certain polyunsaturated long-chain ketones for the treatment of actinic keratosis or squamous cell carcinoma. The invention also relates to methods of treating actinic keratosis or squamous cell carcinoma in patients comprising administration of the compounds of the invention to the patient.

Actinic keratoses, also known as solar keratoses, are pre-cancerous lesions, which occur on skin that has suffered chronic damage from ultraviolet radiation, commonly in the form of sunlight. They typically appear as thick, scaly patches of skin, which have a rough texture and which may vary in colour from red, tan, white or pink.

Sun-exposed areas of the body such as the head, neck, ears and hands are the most commonly involved sites. As they generally result from significant exposure to UV radiation, actinic keratoses are more common in older patients, particularly those over 40 years old. Most patients typically have multiple actinic keratoses. In addition, they are significantly more prevalent in those with fair skin than in those with darker skin.

Exposure of the skin to UV radiation, from the sun or from other sources, results in mutations to the DNA of epidermal keratinocytes which may cause proliferation and expansion of the mutated cells. UV radiation is also known to increase inflammatory markers such as arachidonic acid, as well as other molecules associated with inflammation. The combination of mutated keratinocytes and an environment comprising increased inflammatory markers can ultimately lead to the growth of actinic keratoses.

Current treatments for actinic keratoses include cryotherapy, surgical removal, photodynamic therapy, and topical chemotherapy. It is important that actinic keratoses are accurately and promptly diagnosed and treated, as they can develop into squamous cell carcinoma (SCC). It has been reported that up to 10% of actinic keratoses may develop into squamous cell carcinomas if left untreated, and the majority of SCCs are derived from actinic keratoses.

The present inventors therefore sought alternative and/or combinatorial treatments for this condition.

It has been observed that treatment with non-steroid anti-inflammatory drugs (NSAIDS) that target the arachidonyl cascade may reduce cancer progression (Johannesdottir, S. A., et al., Nonsteroidal anti-inflammatory drugs and the risk of skin cancer: a population-based case-control study. Cancer, 2012. 118 (19): p. 4768-76; Gonzalez-Periz, A. and J. Claria, New approaches to the modulation of the cyclooxygenase-2 and 5-lipoxygenase pathways. Curr Top Med Chem, 2007. 7 (3): p. 297-309).

The present inventors postulate that cytosolic phospholipase A2 group IVa (cPLA2α) enzyme may also be involved in the pathogenesis of actinic keratosis, and in the progression of actinic keratosis into squamous cell carcinoma. The phospholipases A2 enzymes are a group of lipases that by hydrolysis release unsaturated fatty acids from the sn2 position of membrane phospholipids. Once released, the fatty acids are converted by various enzymes into biologically important signalling molecules. Cytosolic group IVa PLA2 (cPLA2α) is pivotal in inflammation; it is activated by intracellular calcium and by phosphorylation in response to stimuli such as pro-inflammatory cytokines and mitogenic growth factors. cPLA2α is selective for AA-containing acyl chains in vitro, and is considered a central enzyme in AA-derived eicosanoid production. The release of arachidonate from phospholipids initiates the arachidonate cascade leading to the synthesis of eicosanoids such as prostaglandins. Eicosanoids are important in a variety of physiological processes and play a central role in inflammation. Elevated levels of arachidonic acid, eicosanoids and other bioactive lipid mediators are reported in inflammatory dermatoses. In particular, the arachidonic acid-derived eicosanoid PGE2 is believed to be an important mediator in the proliferation of keratinocytes and the development of actinic keratosis. The present inventors also realise that a chronic inflammatory microenvironment is now recognized as promoting such proliferation. In this regard, it is noted that such an environment is considered to be a hallmark of cancer, promoting the initiation and growth of abnormal cells. Hence, bioactive lipids may represent a link between inflammation and actinic keratosis, and may be involved in the progression of actinic keratosis into squamous cell carcinoma.

The COX-2/PGE2 pathway is postulated to be important both in the development of actinic keratosis and progression to squamous cell carcinoma (recently reviewed in Thomas G J, Herranz P, Cruz S B, Parodi A. Treatment of actinic keratosis through inhibition of cyclooxygenase-2: Potential mechanism of action of diclofenac sodium 3% in hyaluronic acid 2.5. Dermatol Ther. 2019;32 (3)).

COX-2/PGE2 and PAF are additionally both implicated as mediators of UV-induced immunosuppression, which is considered critical both to the development of actinic keratosis and progression to squamous cell carcinoma (Liu B, Qu L, Yan S. Cyclooxygenase-2 promotes tumor growth and suppresses tumor immunity. Cancer Cell Int. 2015; 15:106. Published 2015 Nov. 5; Damiani E, Ullrich S E. Understanding the connection between platelet-activating factor, a UV-induced lipid mediator of inflammation, immune suppression and skin cancer. Prog Lipid Res. 2016;63:14-27.)

Overexpression of cPLA2 and COX-2 is also postulated in squamous cell carcinoma (Zhang S, Du Y, Tao J, Wu Y, Chen N: Expression of Cytosolic Phospholipase A2 and Cyclooxygenase 2 and Their Significance in Human Oral Mucosae, Dysplasias and Squamous Cell Carcinomas. ORL 2008;70:242-248; Kuźbicki Ł, Lange D, Stanek-Widera A, Chwirot B W. Different expression of cyclooxygenase-2 (COX-2) in selected nonmelanocytic human cutaneous lesions. Folia Histochem Cytobiol. 2011;49 (3):381-8)

It is therefore possible that inhibition of the cPLA2α enzyme, the limiting factor for arachidonic acid and lysophopholipid release and availability, could have potential in reducing the inflammatory environment and inhibiting immunosuppression involved in promoting the formation of actinic keratosis and the development into squamous cell carcinoma due to reduced production of bioactive lipids such as PGE2 and PAF.

An NSAID is currently used in the treatment of actinic keratosis (Wolf J E Jr, Taylor J R, Tschen E, Kang S. Topical 3.0% diclofenac in 2.5% hyaluronan gel in the treatment of actinic keratoses. Int J Dermatol. 2001 November;40 (11):709-13).

The present inventors have surprisingly found that:
1) The use of compounds defined herein block the FBS-stimulated release of PGE2 from HaCaT cells.
2) Compounds defined herein also inhibits the growth of HaCaT cells in the presence and in the absence of FBS.

The compounds proposed for use in this invention have been disclosed before, for example, in EP-A-1469859, e.g. for the treatment of psoriasis, which is a skin condition but is not related to actinic keratosis or squamous cell carcinoma. Psoriasis has very different biochemistry/immunology than actinic keratosis. EP-A-2925326 describes the use of compounds of the invention for the treatment of dermatitis.

The compounds have also been suggested, in EP-3148519, for the treatment of skin cancer but not specifically squamous cell carcinoma or actinic keratosis.

The present inventors have now shown experimentally that the compounds of the invention have utility in the treatment of actinic keratosis through a series of valuable biochemical processes, not limited to the anti-inflammatory effects of cPLA2 inhibition. It is the ability of these compounds to affect multiple biochemical processes, including cell proliferation, viability and possibly differentiation (cell fate) that makes them attractive in the treatment of actinic keratosis, and in the treatment and specifically the prevention of squamous cell carcinoma.

### SUMMARY OF INVENTION

Thus, viewed from one aspect the invention provides a compound of formula (I)

R—S—CH₂—CO—CF₃ (I)
wherein R is a C₁₀₋₂₄ unsaturated hydrocarbon group comprising at least 4 non-conjugated double bonds; or a salt thereof;
for use in the treatment of actinic keratosis.

Viewed from another aspect the invention provides a method of treating actinic keratosis comprising administering to an animal, preferably a mammal, in need thereof, e.g. human, an effective amount of a compound of formula (I)

R—S—CH₂—CO—CF₃ (I)
wherein R is a C₁₀₋₂₄ unsaturated hydrocarbon group comprising at least 4 non-conjugated double bonds; or a salt thereof.

Viewed from another aspect the invention provides use of a compound of formula (I) or a salt thereof as hereinbefore described for use in the manufacture of a medicament for treating actinic keratosis.

Viewed from a further aspect, the invention provides a compound of formula (I)

R—S—CH₂—CO—CF₃ (I)
wherein R is a C₁₀₋₂₄ unsaturated hydrocarbon group comprising at least 4 non-conjugated double bonds; or a salt thereof;
for use in the treatment of squamous cell carcinoma.

Viewed from a further aspect, the invention provides a method of treating squamous cell carcinoma comprising administering to an animal, preferably a mammal, in need thereof, e.g. human, an effective amount of a compound of formula (I):

R—S—CH₂—CO—CF₃ (I)
wherein R is a C₁₀₋₂₄ unsaturated hydrocarbon group comprising at least 4 non-conjugated double bonds; or a salt thereof.

### DETAILED DESCRIPTION

This invention involves the use of compounds of formula (I) or a salt thereof in the treatment of actinic keratosis or squamous cell carcinoma.

### Actinic Keratosis and Squamous Cell Carcinoma

This invention targets actinic keratosis and squamous cell carcinoma. As noted above, actinic keratoses are pre-cancerous lesions which occur on sun exposed areas of the skin. These generally measure between about 2 to about 6 mms in diameter, and can range significantly in colour.

Clinical variants of actinic keratosis include: classic (or common), hypertrophic (or hyperkeratotic), atrophic, actinic keratosis with cutaneous horn, pigmented actinic keratosis, actinic cheilitis, and Bowenoid actinic keratosis. Unless explicitly indicated otherwise, the methods described herein are applicable to all clinical variants, including those listed herein.

Actinic keratosis lesions possess many of the same cellular changes observed in the skin cancer squamous cell carcinoma (SCC), and can develop into SCCs if left untreated. SCC is the second most common form of skin cancer, characterized by abnormal, accelerated growth of squamous cells. SCCs can appear as scaly red patches, open sores, rough, thickened or wart-like skin, or raised growths with a central depression. At times, SCCs may crust over, itch or bleed. Untreated SCCs can become invasive, grow into deeper layers of skin and spread to other parts of the body. Accordingly, it is desirable to prevent the development of SCCs where possible.

As the majority of SCCs are derived from actinic keratoses, it can be seen that the effective treatment of actinic keratosis also constitutes an effective prevention of SCCs. The methods of treating actinic keratosis and compounds for use in treating actinic keratosis which are disclosed herein may therefore also be applied to the prevention of SCCs. Alternatively, all references herein to the treatment of actinic keratosis, may be additionally read as references to the prevention of squamous cell carcinoma.

### Compounds of the Invention

The invention relies on a compound of formula (I)

R—S—CH₂—CO—CF₃ (I)
wherein R is a C₁₀₋₂₄ unsaturated hydrocarbon group comprising at least 4 non-conjugated double bonds; or a salt thereof.

The group R preferably comprises 5 to 9 double bonds, preferably 5 or 8 double bonds, e.g. 5 to 7 double bonds such as 5 or 6 double bonds. These bonds should be non-conjugated. It is also preferred if the double bonds do not conjugate with the carbonyl functionality (CO).

The double bonds present in the group R may be in the cis or trans configuration however, it is preferred if the majority of the double bonds present (i.e. at least 50%) are in the cis configuration. In further advantageous embodiments all the double bonds in the group R are in the cis configuration or all double bonds are in the cis configuration except the double bond nearest the carbonyl group which may be in the trans configuration.

The group R may have between 10 and 24 carbon atoms, preferably 12 to 20 carbon atoms, especially 17 to 19 carbon atoms.

The R group is preferably linear. It preferably derives from a natural source such as a long chain fatty acid or ester. In particular, the R group may derive from AA, EPA or DHA.

The following compounds are highly preferred for use in the invention:

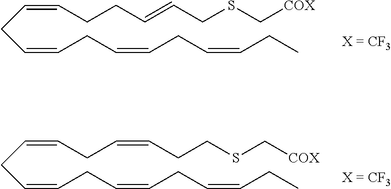

Where possible, the compounds of the invention can be administered in salt form. Preferably however, no such form is used.

Compounds of formula (I) may be manufactured using known chemical synthetic routes, e.g. as in EP-A-2925326 or PCT/EP2016/051456. It is convenient to begin synthesis from the commercially available compounds arachidonic acid (AA), EPA (all-Z-eicosa-5,8,11,14,17-pentaenoic acid) or DHA (all-Z-docosa-4,7,10,13,16,19-hexaenoic acid). Conversion of the acid functionality of these compounds into a —COCF₃ group can be achieved readily, e.g. by converting the carboxylic acid into its corresponding acid chloride and reacting the same with trifluoroacetic anhydride in the presence of pyridine.

Introduction of an S atom into the carbon chain is also achieved readily. Conveniently, for example, the starting acid is reduced to an alcohol and, if required, converted to the corresponding thiol. The nucleophilic thiol may then be reacted with a group such as BrCH₂COCF₃ thereby introducing the carbonyl and electron trifluoromethyl species. Complete synthetic protocols may be found in J. Chem. Soc., Perkin Trans 1, 2000, 2271-2276 or J. Immunol., 1998, 161, 3421.

The compounds of the invention are proposed primarily for use in the treatment of, inter alia, actinic keratosis.

By treating or treatment is meant at least one of:
(i). preventing or delaying the appearance of clinical symptoms of the disease developing in a mammal;
(ii). inhibiting the disease i.e. arresting, reducing or delaying the development of the disease or a relapse thereof or at least one clinical or subclinical symptom thereof, or
(iii). relieving or attenuating one or more of the clinical or subclinical symptoms of the disease.

It is particularly preferred to use the compounds of the invention to prevent squamous cell carcinoma. It is particularly preferred to use the compounds of the invention to relieve or attenuate one or more of the clinical symptoms of the actinic keratosis.

The benefit to a subject to be treated is either statistically significant or at least perceptible to the patient or to the physician. In general a skilled person can appreciate when “treatment” occurs. It is particularly preferred if the compounds of the invention are used therapeutically, i.e. to treat a condition which has manifested rather than prophylactically. It may be that the compounds of the invention are more effective when used therapeutically than prophylactically.

The compounds of the invention can be used on any animal subject, in particular a mammal and more particularly to a human or an animal serving as a model for a disease (e.g., mouse, monkey, etc.).

In order to treat a disease an effective amount of the active agent needs to be administered to a patient. A “therapeutically effective amount” means the amount of a compound that, when administered to an animal for treating a state, disorder or condition, is sufficient to effect such treatment. The “therapeutically effective amount” will vary depending on the compound, the disease and its severity and the age, weight, physical condition and responsiveness of the subject to be treated and will be ultimately at the discretion of the attendant doctor.

It may be that to treat conditions according to the invention that the compound of formula (I) has to be reapplied at certain intervals. Suitable dosage regimes can be prescribed by a physician.

While it is possible that, for use in the methods of the invention, a compound of formula I may be administered as the bulk substance, it is preferable to present the active ingredient in a pharmaceutical formulation, for example, wherein the agent is in admixture with a pharmaceutically acceptable carrier selected with regard to the intended route of administration and standard pharmaceutical practice.

The term “carrier” refers to a diluent, excipient, and/or vehicle with which an active compound is administered. The pharmaceutical compositions of the invention may contain combinations of more than one carrier. Such pharmaceutical carriers are well known in the art. The pharmaceutical compositions may also comprise any suitable binder(s), lubricant(s), suspending agent(s), coating agent(s), and/or solubilizing agent(s) and so on.

The compositions can also contain other active components, e.g. other drugs for the treatment of actinic keratosis or squamous cell carcinoma.

The active agent of the invention may therefore be combined with steroids or barrier materials (such as zinc oxide).

It will be appreciated that pharmaceutical compositions for use in accordance with the present invention may be in the form of oral, parenteral, transdermal, sublingual, topical, implant, nasal, or enterally administered (or other mucosally administered) suspensions, capsules or tablets, which may be formulated in conventional manner using one or more pharmaceutically acceptable carriers or excipients.

However, for the treatment of actinic keratosis, the compositions of the invention will preferably be administered orally or ideally topically. The compound may therefore be provided in the form of an ointment, cream, salve, foam or gel.

The pharmaceutical compositions of the invention may contain from 0.01 to 99% weight-per volume of the active material.

A therapeutically effective amount of the compound of the present invention can be determined by methods known in the art. The therapeutically effective quantities will depend on the age and on the general physiological condition of the patient, the route of administration and the pharmaceutical formulation used. The therapeutic doses will generally be between about 1.0 and 200 mg/day and preferably between about 3.0 and 150 mg/day. Other ranges may be used, including, for example, 5.0-50 mg/day, 5.0-30 mg/day. A typical range is 10-200 mg/day.

Administration may be once a day, twice a day, or more often, and may be decreased during a maintenance phase of the disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art.

The compounds of the invention may be used to treat squamous cell carcinoma in combination with other known pharmaceuticals for said purpose and this forms a further aspect of the invention. In particular, a combination with Imiquimod, 5-Fluorouracil (5-FU) or Erivedge™ (vismodegib) is contemplated.

Alternatively, the compounds of the invention might be used in combination with radiotherapy, cryotherapy, phototherapy, laser therapy or radiation therapy. The invention is described further below with reference to the following non-limiting examples and figures.

### DESCRIPTION OF FIGURES

FIG. 1 shows in (A) and (B) the cell count of HaCaT cells cultured in DMEM-0.5% FBS (A) or 10% FBS (B) in the absence or presence of Compound A (the legend shows the concentrations of Compound A in μM). FIG. 1 (C) shows a dose response curve for the 96 hour time point for both experiments.

FIG. 2 shows the concentration of PGE2 following the stimulation of HaCaT cells which had serum deprived for 24 h by addition of 10% FBS or CTRL (0.5% FBS) in the absence or presence of Compound A (5 μM) for 24 h. *P<0.05 vs CTRL, #p<0.05 vs 10% FBS.

FIG. 3 shows that AVX001 inhibits the growth, proliferation and viability of primary keratinocytes. Growth curve: cells were treated with vehicle or AVX001 and counted by automated imaging every day for upto 6 days. Data are the means of 2-3 experiments. Viability assay: resazurin assays were used to measure cell viabilty in cells cultivated for 3 days and treated for a further 3 days with either Vehicle or AVX001. The data are a representative experiment, where data points are the mean±stdev for 6 technical replicates. The average IC50 of AVX001 from 3 experiments was 3.71 μM±0.7. Proliferation index: the proliferating/total cells after 24 hours treatment with vehicle or AVX001. Data are the mean±SEM for 3 experiments. *p<0.05, ***p<0.001 versus vehicle (one-way ANOVA).

FIG. 4 shows that AVX001 inhibits the viability of A431 cutaneous squamous cell carcinoma cells. A. Representative images of A431 cells treated AVX001. Phase contrast images were taken at 20× magnification following 2 days of culture with either vehicle or AVX001. B. Cell viability assay performed in 6 replicate wells. Individual data point for 6 replicates are shown. Curve fitting by non-linear regression was used to calculate IC50s.

FIG. 5. BIRC5 is a target of cPLA2α inhibition in HaCaT keratinocytes. qPCR analysis of BIRC gene expression in 3D cultures of HaCaT cells treated with either vehicle or AVX001 (5 μM) for 12 days. Data are the mean normalized relative quantities (NRQ)±SEM. *p<0.05 (unpaired t-test).

### EXAMPLES

The following compound was used in the Experiments:

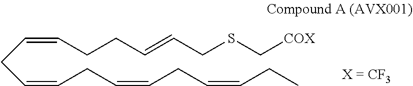

### Example 1

### Materials

Cell culture media, and chemicals were purchased from Sigma-Aldrich unless stated otherwise. The fluoroketone was stored at −80° C. as a 20 mM stock solution in DMSO under argon gas to minimize oxidation.

### Maintenance of HaCaT Keratinocytes

The spontaneously immortalized skin keratinocyte cell line HaCaT was used. These cells are commonly used to study proliferative responses in dermatology, express EGFR and can proliferate both independently of, and in response to stimulation with growth factors. HaCaT were maintained in DMEM supplemented with 5% (v/v) FBS, 0.3 mg/ml glutamine, and 0.1 mg/ml gentamicin (DMEM-5) at 37° C. with 5% CO2 in a humidified atmosphere at sub-confluency to prevent differentiation. Treatments were carried out in DMEM supplemented with 0.5% (v/v) FBS, 0.3 mg/ml glutamine (DMEM-0.5)

### Cell Growth Assays

HaCaT were seeded in 96 well plates in DMEM-5 at a density of 3000 cells per well. After 24 hours, 4 brightield images were captured per well using the Biotek Cytation 5 multimode plate reader equiped with a 10× objective. Each field was captured using both the automatic autofocus, and with an offset to generate an out-of-focus image that was optimal for accurate detection and counting of the number of cells per field. The media was then replaced with DMEM-0.5 in the absence or presence of Compound A at the doses indicated; 6 wells were used per treatment. After 90 min, a final concentration of 10% FBS was added to half of the wells and plates were incubated at 37° C., 5% CO2. Brightfield images for cell counting were taken at 24 h intervals for 6 days, with the media and treatments being replaced after 3 days.

### Enzyme-Linked Immunoassay Detection of PGE2

HaCaT cells were seeded in 24-well plates in DMEM-5 at a density of 20 000 cells per well. After 3 days the cells were serum deprived for 24 hours in DMEM-0.5, then pre-treated with Compound A or vehicle (DMSO) for 90 min before the addition of a final concentration of 10% FBS. After a further 24 h the supernatant was removed and PGE2 levels were measured by enzyme-linked immunosorbent assay (EIA) (Cayman #514435) according to the manufacturers' protocols. Cell supernatants were assayed undiluted. Supernatants were hybridized over-night, and the enzymatic conversion of substrate was read at OD420 nm. Data were processed using a 4-parameter logistic fit model.

### Results

Growth of HaCaT Cells Under a Range of Conditions is Inhibited by cPLA2α Inhibitor Compound A

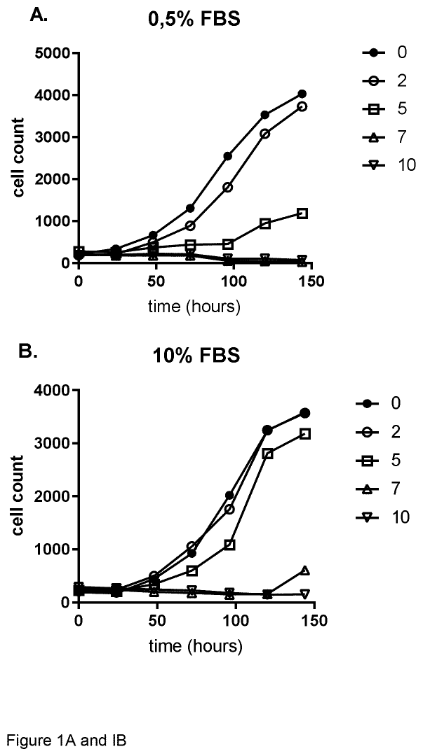

Compound A treatment inhibited the growth of HaCaT grown both under serum-deprivation (0.5% FBS) (FIG. 1A) and in the presence of 10% FBS (FIG. 1B). At 96 hours post-treatment, Compound A inhibited the growth of the HaCaT cells with an average IC50 of 3.2 μM in 0.5% FBS and 5 μM in 10% FBS (FIG. 1C and table 1).

HaCaT cells were cultured in DMEM-0.5% FBS (A) or 10% FBS (B) in the absence or presence of Compound A (concentrations in μM). Cell counts were recorded every 24 hours for 6 days. The data shown below is the mean of 3 technical replicates and the experiment was repeated twice. Non-linear regression was used to calculated IC50 values from the dose response curves (FIG. 1C), which are shown for each of the bioreplicates (REP1 and REP2).

TABLE 1
IC50 values at 96 hours post-treatment

| | REP1 | REP2 |
|---|---|---|
| 0.5% FBS | 3.4 μM | 2.9 μM |
| 10% FBS | 5.1 μM | 5.0 μM |

The release of PGE2 from HaCaT Cells is Blocked by cPLA2α Inhibitor Compound A

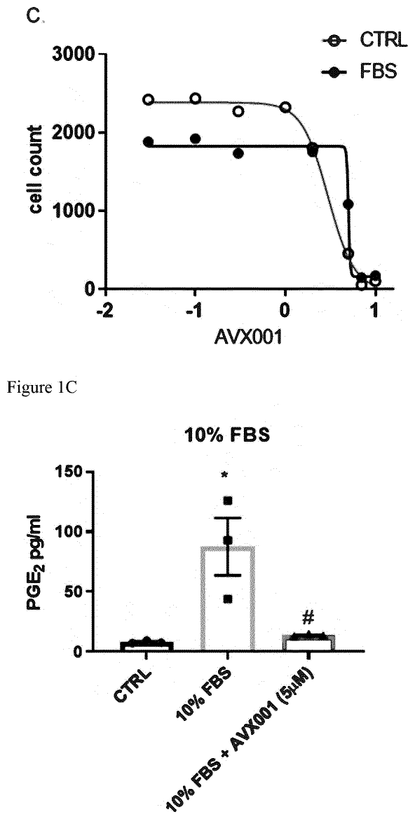

In HaCaT cells deprived of serum for 24 hours, the addition of 10% FBS for 24 h induced the release of PGE2. PGE2 levels were analysed by enzyme-linked immunoassay. The data shown in FIG. 2 is the mean of 3 independent experiments. Pre-incubation with Compound A (5 μM) completely blocked the FBS-stimulated release of PGE2, indicating that activation of cPLA2α is important driver of FBS stimulated PGE2 release. This mechanism may be important for regulating the proliferation of keratinocytes.

### Example 2

### Methods

### Culture and Experiments in Primary Human Epidermal Keratinocytes

Human neonatal epidermal keratinocytes pooled from 4-6 individuals (Thermofisher #A13401) were grown on collagen coated flasks in Epilife medium (M-EPI-500-CA) supplemented with supplement S7 according to the suppliers' instructions. The media was replaced every 2 days and cells were sub-cultured before reaching confluence. For experiments, cells were plated into 96 well plates at a density of 2500 cells per cm². For growth assays, after 24 hours the cells were treated with AVX001 at the indicated doses and the growth was monitored by counting cells every day for 6 days using automated imaging and analysis using a Cytation 5 multimode imaging plate reader (Biotek Inc.). For cell viability assays, the cells were treated with AVX001 at the indicated doses on day 4 after plating and incubated for a further 3 days before the addition of resazurin reagent for 2 hours prior to reading the fluorescence at 590 nm using a Cytation 5 multimode imaging plate reader (Biotek Inc.) as an indicator of the number of live cells. For cell proliferation assays, the media was changed to Epilife media without supplement S7 at day 5 after plating. The next day the cells were treated with AVX001 at the indicated doses for 24 hours. EdU was added for 2 hours before the cells were fixed in 4% paraformaldehyde and actively proliferating cells were quantified using The ClickIT EdU Alexafluor 594 HCS assay (Thermofisher Scientific). The protocol for permeabilization and staining was performed according to the manufacturers' instructions. The cells were imaged at 10× magnification using a Cytation 5 multimode imaging plate reader (Biotek Inc.) with DAPI and TRITC filter sets to image the HCS nuclear mask and EdU respectively. Total and EdU positive nuclei were counted using the automated analysis software CellProfiler and presented as the proliferative index (EdU positive/total nuclei per image).

### Culture and Experiments in A431 Cutaneous Squamous Cell Carcinoma Cells and HaCaT Cells

A431 cutaneous squamous cell carcinoma cells and HaCaT immortalized keratinocytes were cultured in DMEM supplemented with 5% FBS. For experiments, the cells were plated in 96 well plates at a density of 3000 cells per well in DMEM supplemented with 5% FBS. After 2 days, the media was changed to DMEM supplemented with 0.5% FBS in the presence of vehicle (0.1% DMSO) or AVX001 for a further 2 days. Resazurin (10 μL per well) was added for 2 hours prior to reading the fluorescence at 590 nm using a Cytation 5 multimode imaging plate reader (Biotek Inc.) as an indicator of the number of live cells.

### Analysis of Gene Expression by Quantitative PCR

HaCaT keratinocytes were maintained and grown in 3D organotypic cultures. RNA was extracted from the cultures using the RNeasy minikit (Qiagen) and reverse transcription was performed using the Quantitect reverse transcription kit according to the manufacturers protocol. Quantitative PCR was performed with SYBR green master mix (Roche) using a LightCycler 96 instrument from Roche. Cq values were calculated in LinReg PCR (version 2017.1) and quantification relative to three reference genes (GAPDH, HPRT1, and RPS18) was carried out using qbase+ software, version 3.0 (Biogazelle, Zwijnaarde, Belgium—www.qbaseplus.com)

| Gene | Sense primer | Antisense primer |
|---|---|---|
| BIRC5 | 5′-ATTCGTCCGGT | 5′-CACGGCGCACTT |
| | TGCGCTTTCC-3′ | TCTTCGCAG-3′ |
| BIRC2 | 5′-CAGCCTGAGCA | 5′-CAAGCCACCATC |
| | GCTTGCAA-3′ | ACAACAAAA-3′ |
| BIRC3 | 5′-CCGTCAAGTTC | 5′-AAGCCCATTTCC |
| | AAGCCAGTTACC | ACGGCAGC-3′ |
| | C-3′ | |
| BIRC4 | 5′-AGTGGTAGTCC | 5′-CCGCACGGT |
| | TGTTTCAGCATC | ATCTCCTTCA-3′ |
| | A-3′ | |

### Results and Discussion

### AVX001 Inhibits the Viability of Primary Human Keratinocytes and Transformed Keratinocytes in Culture

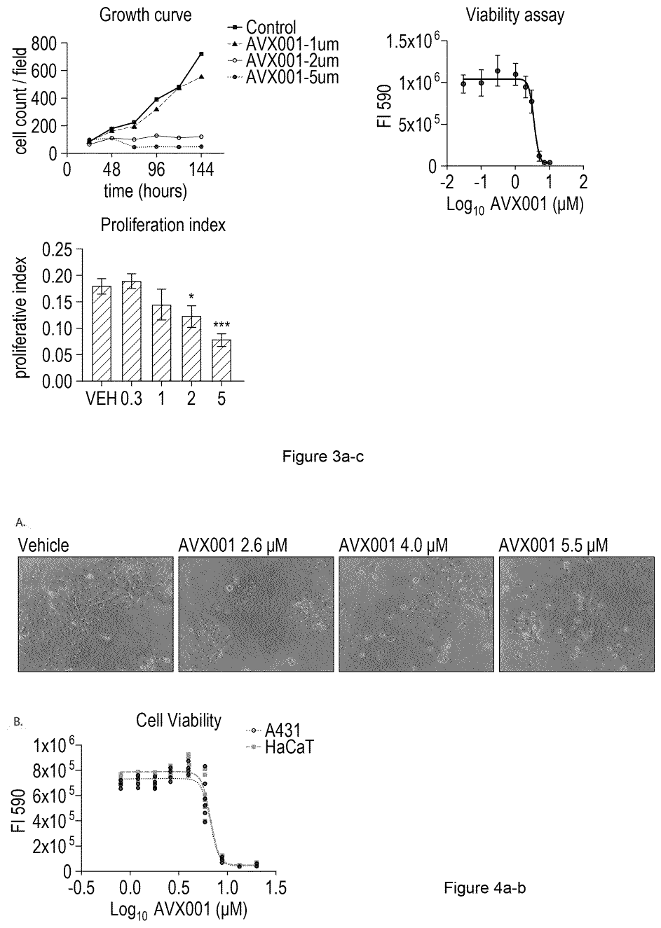

Previously we have shown that AVX001 can suppress the growth and proliferation of immortalized HaCaT keratinocytes in both 2D and 3D culture and can inhibit responses to growth factors and FBS. To test whether AVX001 can inhibit proliferation in primary keratinocytes, we cultured commercially available primary human epidermal keratinocytes (HEKs) investigated the effects of AVX001 on their growth, viability and proliferation. At a concentration of 2 μM, AVX001 prevented the proliferation of the HEKs (FIG. 3a; growth curve) and inhibited their viability with an average IC50 of 3.7 μM±0.7 (FIG. 3b; viability assay). Proliferation assays indicate that at these concentration (2-5 μM), AVX001 significantly reduced the proportion of actively proliferating cells in the population (FIG. 3c; proliferation assay). This supports data from the immortalized HaCaT cells suggesting that cPLA2α activity is important for the proliferation/survival of keratinocytes.

To test whether AVX001 is effective at inhibiting growth of transformed keratinocytes we cultured the cutaneous squamous cell carinoma A431 cell line and measured cell viability following treatment with increasing doses of AVX001. AVX001 reduced the viability of A431 cells, demonstrated by the reduction in the number of attached cells showin in FIG. 4A and reduced metabolism as indicated by resazurin assay shown in FIG. 4B. As a comparison we also repeated viability studies previously carried out in HaCaT cells. AVX001 reduced the viability of A431 cells with an IC50 of 6.7 μM, which was equivalent to the effect of the compound in HaCaT cells.

### AVX001 Supresses the Expression of BIRC5, the Gene Encoding Survivin

While it is clear that cPLA2α activity is important for the growth and proliferation of multiple cell types, the signaling pathways and effector proteins are likely to be dependent upon the origin and stage of development of the cells/tissue. In order to investigate which pathways/effectors could be important for cPLA2α dpendent proliferation of keratinocytes, we extracted RNA from HaCaT cells cultured in 3D in the presence or absence of AVX001, and measured the expression of members of the baculovirus IAP repeat containing (BIRC) family of antiapoptotic proteinss, which have been shown to be involved in the development of hyperproliferative disorders, including in skin.

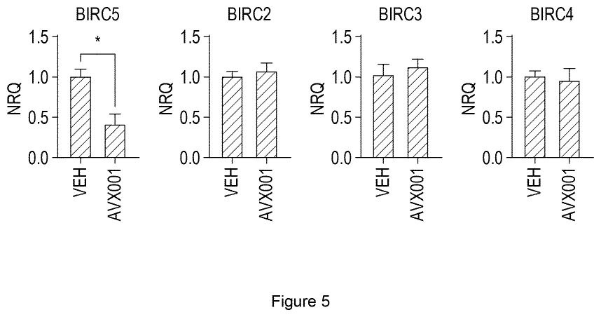

We demonstrated that the HaCaT 3D cultures treated with AVX001 had significantly lower levels of BIRC5 gene expression than those treated with the vehicle control, whereas expression of BIRC2, BIRC3 and BIRC4 were unaffected (FIG. 5). BIRC5 is the gene that encodes survivin, a protein with established roles in cell cycle progression and protection from apoptosis in keratinocytes. Survivin is overexpressed in cancers and inflammatory skin disorders, and is involved in hyperproliferation and skin tumour development. BIRC5 is also a known target of PGE2/EP2/STAT3 signaling in UV-stimulated keratinocytes and targeting survivin can inhibit the growth of melanoma skin cancers in mice. Down-regulation of survivin by AVX001 treatment would thus be predicted to impair proliferation and survival of keratinocytes and may thus present a putative mechanism of action for AVX001 in actinic keratosis and squamous cell carcinoma.

1-8. (canceled)

9. A method of treating actinic keratosis or squamous cell carcinoma comprising administering to an animal, an effective amount of a compound of formula (I):
R—S—CH₂—CO—CF₃ (I)
wherein R is a C₁₀₋₂₄ unsaturated hydrocarbon group comprising at least 4 non-conjugated double bonds; or a salt thereof.

10. (canceled)

11. The method of claim 9, wherein the animal is a mammal.

12. The method of claim 11, wherein the mammal is a human.

13. The method of claim 9, wherein said hydrocarbon group R has 5 to 7 double bonds.

14. The method of claim 9, wherein in said hydrocarbon group R, no double bond is conjugated with the carbonyl group.

15. The method of claim 9, wherein in said hydrocarbon group R all double bonds are in the cis configuration or wherein in said hydrocarbon group all double bonds are in the cis configuration except the double bond nearest the carbonyl.

16. The method of claim 9, wherein the R group comprises 17 to 19 carbon atoms.

17. The method of claim 9, where the compound of formula (I) is

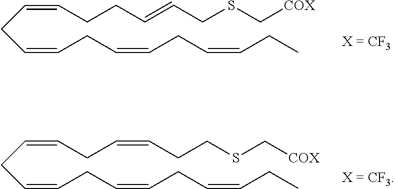

18. The method of claim 9, wherein the compound is administered orally or topically.


SEQUENCE LISTING (8 sequences)

SEQ ID NO: 1 - DNA, 21 bp, Artificial Sequence
  BIRC5 sense primer
attcgtccggttgcgctttcc

SEQ ID NO: 2 - DNA, 21 bp, Artificial Sequence
  BIRC5 antisense primer
cacggcgcactttcttcgcag

SEQ ID NO: 3 - DNA, 19 bp, Artificial Sequence
  BIRC2 sense primer
cagcctgagcagcttgcaa

SEQ ID NO: 4 - DNA, 21 bp, Artificial Sequence
  BIRC2 antisense primer
caagccaccatcacaacaaaa

SEQ ID NO: 5 - DNA, 24 bp, Artificial Sequence
  BIRC3 sense primer
ccgtcaagttcaagccagttaccc

SEQ ID NO: 6 - DNA, 20 bp, Artificial Sequence
  BIRC3 antisense primer
aagcccatttccacggcagc

SEQ ID NO: 7 - DNA, 24 bp, Artificial Sequence
  BIRC4 sense primer
agtggtagtcctgtttcagcatca

SEQ ID NO: 8 - DNA, 19 bp, Artificial Sequence
  BIRC4 antisense primer
ccgcacggtatctccttca